SEQUENCE LISTING (11 sequences)

SEQ ID NO: 1 - DNA, 60 bp, artificial sequence
  chemically synthetized
ggggctagggttcctccgcgttatcatgctaagatttctcctatggtgaagggggccgct

SEQ ID NO: 2 - protein, 15 aa, artificial sequence
  chemically synthetized
RVPPRYHAKISPMVN

SEQ ID NO: 3 - protein, 10 aa, artificial sequence
  chemically synthetized
SFGSGFGGGY

SEQ ID NO: 4 - protein, 15 aa, artificial sequence
  chemically synthetized
RVPPRYHAKISPMVA

SEQ ID NO: 5 - protein, 6 aa, artificial sequence
  chemically synthetized
VPPRYH

SEQ ID NO: 6 - protein, 8 aa, artificial sequence
  chemically synthetized
AKISPMVN

SEQ ID NO: 7 - protein, 8 aa, artificial sequence
  chemically synthetized
AKISPMVA

SEQ ID NO: 8 - protein, 14 aa, artificial sequence
  chemically synthetized
RVPPRYHAKISPMV

SEQ ID NO: 9 - protein, 10 aa, artificial sequence
  chemically synthetized
RVPPRYHAKI

SEQ ID NO: 10 - protein, 7 aa, artificial sequence
  chemically synthetized
RVPPRYH

SEQ ID NO: 11 - protein, 14 aa, artificial sequence
  chemically synthetized
VPPRYHAKISPMVN

(19) United States
(12) Patent Application Publication
(10) Pub. No.: US 2009/0142364 A1
(43) Pub. Date: Jun. 4, 2009

(54) Method for finding novel peptide immunostimulatory adjuvants, compositions and methods of use

(76) Inventors: Fedor Alexandrovich Brovko, Pushchino (RU); Timo Kalevi Korpela, Turku (FI); Alexander Georgievich Laman, Pushchino (RU); Galina Vladimirovna Malakhova, Moscow (RU); Vladimir Andreevich Nesmeyanov, (US); Igor Leonidovich Rodionov, Pushchino (RU); Anna Olegovna Shepelyakovskaya, Pushchino (RU)

Correspondence Address: DODDS & ASSOCIATES, 1707 N STREET NW, WASHINGTON, DC, 20036, US

(21) Appl. No.: 12/229,968

(22) Filed: Aug. 27, 2008

Related U.S. Application Data
(60) Provisional application No. 60/966,858, filed on Aug. 30, 2007.

(51) Int. Cl. A61K 39/00; C40B 30/04; C07K 7/06; C07K 7/08; A61K 38/08; A61K 38/10; C07H 21/00; C12N 15/63; C12N 5/00

(52) U.S. Cl. 424/185.1; 506/9; 530/326; 530/328; 530/327; 530/329; 536/23.1; 435/320.1; 435/325; 514/16; 514/15; 514/14

(57) ABSTRACT

The invention describes production of an adjuvant with biological activities related to N-acetylglucosaminyl-β1-4-N-acetylmuramyl-alanyl-D-isoglutamine (GMDP), a commonly used enhancer of the immune response. It was found in the present invention that the drawbacks of GMDP can be avoided by replacing it with a peptide that does not have any direct structural relationship with GMDP.

### PRIORITY

This application claims priority of the provisional patent application No. 60/966,858 filed on Aug. 30, 2007.

### SEQUENCE DATA

This application contains sequence data provided on computer readable diskette and same data on paper.

### FIELD OF INVENTION

The invention is related to medical technology and biotechnology. In particular, the invention relates to a method to produce peptides to be used as immune response modifiers, namely adjuvants, stimulating antibody formation, cellular immune responses and as compounds inducing non-specific resistance to infections. The peptides produced by the method of the invention can be used with vaccines and antigens for production of immune responses, including but not limited to antibody responses.

### BACKGROUND OF THE INVENTION

Production of vaccines and antibodies is one of the economically biggest branches of bioindustry. Both of these technologies involve generation of antibodies by living cells. However, without additional immunostimulatory ingredients or adjuvants, the antibody response is normally weak. Poor antigens can often be converted to immunogenicity by use of adjuvants.

Traditionally, vaccines for humans and animals have been prepared from microorganisms killed by compounds such as formaldehyde or from attenuated microorganisms of low virulence. Newer methods utilize purified proteins of microorganisms, bacterial capsular polysaccharides, or DNA. Nevertheless it is often very difficult to design effective vaccines. For example, in spite of 80 years of investigations, better vaccines for tuberculosis are still needed and all attempts to produce an AIDS vaccine have failed up till now. A satisfactory vaccine must activate both B and T cells. Activation of the latter may be especially difficult (see e.g., Metzler, 2003). Traits related to the immunization techniques are described, for example, in U.S. Pat. No. 4,946,676 Wetzel et al.

For more than sixty years, Freund's complete adjuvant (FCA) has been used to boost antibody response in laboratory animals. E. Lederer's group found that N-acetylmuramyl-alanyl-D-isoglutamine (muramyl dipeptide, MDP) which is a fragment of the bacterial cell wall peptidoglycan, murein, can substitute for the killed mycobacteria in FCA (Ellouz et al. 1974). Aside from the adjuvant effect, MDP induced non-specific resistance to infections, activation of macrophages, and stimulated biosynthesis of certain cytokines (Lederer 1988). Rostovtseva et al. (1981) showed that the repeating unit of murein, N-acetylglucosaminyl-β1-4-N-acetylmuramyl-alanyl-D-isoglutamine (glucosaminylmuramyl dipeptide, GMDP) possessed a stronger immunomodulatory activity than MDP. The adjuvant effect of GMDP is independent of the antigen employed. Based on this glycopeptide, the medicine Licopid was developed for the therapy of secondary immunodeficiency and of certain other infections (Ivanov et al. 1996).

Currently, very few adjuvants are authorized for human use, the most common being aluminium hydroxide and aluminium phosphate. While sufficient for many vaccines, these adjuvants are not as effective as Freund's complete adjuvant or Freund's incomplete adjuvant. Freund's adjuvants are known to have a very undesirable side effect of producing granulomas at injection sites as stated in WO 01/47553 by Zuckerman et al. Muramyl-peptides are being considered as candidate adjuvants for human use.

Despite its favorable properties, GMDP has certain undesirable features as an adjuvant for vaccines, in particular, pyrogenicity. Pyrogens (fever-inducing agents) are especially harmful in vaccines. When producing antibodies in animals, pyrogens are also harmful for the laboratory animals. A number of patents are targeted to the removal of pyrogens from water and materials aimed at production of vaccines. In the present invention it was found that certain relatively short peptides mimicking GMDP's spatial structures can stimulate antibody production as well as induce resistance to infections in a fashion similar to that achieved by GMDP itself, but which do not exhibit an adverse effect exhibited by GMDP itself, namely, pyrogenicity.

In an invention described in U.S. Pat. No. 4,946,676 by Wetzel et al., certain peptides, originating from an antigen, increased the antibody production against the cognate antigen. Such peptides were, however, related to the antigen itself and possibly their effect was similar to the effect achieved by repeated immunization (booster) that is well known in the art. Other peptide adjuvants have also been offered. Their design is frequently based on formation of micelles obtained by chemical introduction of a fatty acid moiety into peptides or by using integrin-type motifs into the peptides (WO 01/47553 by Zuckerman et al.).

A new peptide vaccine against Group B streptococcus has been generated by using the method of phage-display technique (WO 99/33969 by Pincus). The peptide was specially created to mimic capsular polysaccharide of Group B streptococcus. However, the phage-display technique was utilized to produce the antigen rather than adjuvant, as in the present invention. Patent application publication WO 02/13857 by Egyed et al. describes cathelicidins for preparing adjuvants for immunization. However, the adjuvants of the present invention are artificial and not found in nature.

### SUMMARY OF THE INVENTION

The present invention avoids the drawbacks of previously known adjuvants by utilizing the methods, uses and compounds characterized in this specification and in the appended claims. In the present invention, we surprisingly found that the phage-display method can be employed for selecting novel adjuvants for the generation of antibodies. While the phage display technique is a well established method for producing molecules attaining a conformation mimicking the conformation of the original molecules, it could not be anticipated that such molecular mimetics based on these peptides can function as adjuvants, because the adjuvant peptides will be embedded in solutions unfolding the peptide conformations. Moreover, it was surprising that the adjuvant peptides were stable against proteolysis for long periods during generation of immune responses. Furthermore, the peptide generated against the original biological model did not repeat all properties of the model. Namely, the drawback of pyrogenicity was not reproduced in the artificial peptide mimetics.

Consequently, a primary object of the present invention is a peptide having adjuvant activity of N-acetylglucosaminyl-β1-4-N-acetylmuramyl-alanyl-D-isoglutamine (GMDP), wherein said peptide specifically binds to an antibody generated against GMDP. Preferably, the peptide has a sequence RVPPRYHAKISPMVN (SEQ ID NO: 2). The peptide may also be a truncated or chemically modified form thereof involving not less than 7 amino acid residues, provided it retains said adjuvant activity.

In a preferred peptide, the N-terminal residue is positively charged. The peptides of the invention are produced by the process of:
a) generating one or several antibodies against GMDP,
b) contacting a peptide-displaying library with one or several of said antibodies,
c) isolating one or several peptide displaying entities having a displayed peptide which binds to one or several of said antibodies, and
d) selecting from all said peptide displaying entities isolated in step (c) the peptide(s) or peptide fragment(s) to which one or several of said antibodies bind,
e) expressing the selected peptide(s) and, optionally, modifying them by chemical reagents.

A further object of the invention is to provide an isolated nucleic acid sequence, GGG GCT AGG GTT CCT CCG CGT TAT CAT GCT AAG ATT TCT CCT ATG GTG AAG GGG GCC GCT (SEQ ID NO: 1), or a variant thereof which encodes SEQ ID NO: 2, SEQ ID NO: 4, SEQ ID NO: 5, or a variant thereof as disclosed herein, and elongated, degenerated or truncated forms thereof encoding an active peptide adjuvant according to this invention.

Still further objects of the invention are an isolated plasmid comprising the nucleic acid of SEQ ID NO: 1, and an isolated recombinant cell comprising said plasmid.

The peptides of the invention are useful in a method for increasing antibody formation against an antigen, wherein the peptide is administered to a subject simultaneously with or prior to administration of said antigen.

A preferred peptide of the present invention is RVPPRYHAKISPMVN (SEQ ID NO: 2); another preferred peptide according to this invention is RVPPRYHAKISPMVA (SEQ ID NO: 4). Another preferred peptide according to this invention is X₁VPPRYHX₂ (SEQ ID NO:5), wherein X₁ represents a positively charged amino acid, a positively charged protecting group, or both; X₂ represents either no amino acids, one to several amino acids, one or more hydrophobic groups, a fluorescent molecule, the peptide sequence AKISPMVN (SEQ ID NO: 6), the peptide sequence AKISPMVA (SEQ ID NO: 7), and combinations and variations thereof, including conservative amino acid modifications, substitutions, and derivatives, and including salts, and other pharmaceutically acceptable formulations thereof.

### BRIEF DESCRIPTION OF THE DRAWINGS

FIG. 1. Determination of the association constants between antibodies (mAb E6/1.2) and peptides. This was carried out according to Beatty, J. D. et al. (1987). The concentration of phages was 10¹¹ cfu/ml (1) and 5×10¹⁰ cfu/ml. [MA]=1.1×10⁻⁸ M, [MA′]=1.6×10⁻⁸ M. Kₐ=1.6×10⁷M⁻¹.

FIG. 2. Adjuvant activities of RN-peptide analogs with singe Ala substitutions of given amino acid residues. Mice were immunized i.p. with ovalbumin in PBS in the presence of the given peptide (1 μg/mouse). Numbers above the bars depict adjuvant indexes of analogs. The residues, replacement of which had the major impact on the adjuvant activity, are underlined.

FIG. 3. Changes in rectal temperature of rats upon injection of 5.74×10⁻⁷ mole/kg of RN-peptide or GMDP (relative to initial temperature, %): n—the number of animals in groups; *—p<0.05, compared with initial temperature; #—p<0.05, compared with control (GMDP) group.

FIG. 4. Effect of different doses of RN-peptide or GMDP in saline on survival of mice against lethal doses of E. coli. The horizontal axis numbers mean: 1, immediately after infection; 2, 18 h; 3, 27 h; 4, 29 h; 5, 4 h; 6, 50 h; and 7, 72 h.

FIG. 5. Effect of different doses of RN-peptide or GMDP in saline on survival of mice against half-lethal doses of E. coli. The horizontal axis numbers mean: 1, immediately after infection; 2, 18 h; 3, 27 h; 4, 29 h; 5, 4 h; 6, 50 h; and 7, 72 h.

### DETAILED DESCRIPTION OF THE INVENTION

Adjuvant is defined in the present invention as an additive which increases the generation of antibodies against antigen(s), but which is not a part of the antigen and which does not essentially generate antibody production itself. In the present invention the method of producing useful adjuvants is exemplified by the peptide RVPPRYHAKISPMVN (SEQ ID NO: 2), which is termed hereinafter as RN-peptide. Its truncated heptapeptide is termed RH-peptide.

Fever-inducing agents, pyrogens, are produced by bacteria, molds, viruses, and yeasts and they commonly occur in liquids which have been in contact with the microbes.

The term mimotope means a part of a peptide mimetic structure resembling a certain epitope (antigenic determinant) of the original compound and interacting with antibody produced against this compound.

The term immunogenicity refers to the ability to induce adaptive immune responses, including humoral immune responses, cellular immune responses, or both.

The basic embodiment of the present invention is that it is possible to design artificial peptide adjuvants mimicking natural adjuvants retaining immunostimulatory properties, but lacking the undesirable side effects of commonly used adjuvants. It is practically impossible to work out any related adjuvant structures by computer modeling and genetic engineering approaches based on the knowledge of the microbial cell wall structures, or of the structures of other useful adjuvants. Even if any knowledge of the 3-D structures would be available, peptide sequences could not be constructed which could mimic said structures well enough to be biologically active as adjuvants.

In the present invention we approached these problems through an indirect, untraditional way by screening an enormous number of random peptide structures of different lengths and amino acid compositions to identify peptides mimicking molecules known to possess adjuvant activity. Although such a procedure is possible by synthesizing and purifying a very large number of peptides by known chemical methods and testing their activities, this demands a work input impossible in practice.

We found that it is possible to solve this problem by using the phage-display technique for selecting proper peptide structures. Other methods of peptide display known in the art could also be used to similar effect. The technical level in the field is illustrated in Sparks, A. B., et al. (1996). The method requires a random library of peptides of certain length fused to the phage's structural protein. In one embodiment according to the present invention, GMDP was chosen as a model adjuvant. While the general technique of phage display is known, it is not obvious that a product of using that technique would function as an adjuvant. First, non-glycosylated peptides have half-lives of only hours in blood circulation, while formation of antibody-based immune response takes several days. Second, it is possible to create a multitude of molecular mimetics for natural adjuvant molecules by the phage-display technique. All of them will not necessarily be biologically active; since it is not known which part of the adjuvant will trigger the immune response.

It is of special importance that effective screening methods exist for selecting biologically active peptides among the enormous number of inactive peptide structures within the phage-display library. Therefore, the second embodiment of the present invention is that monoclonal antibodies can be effectively exploited for the screening. While it can be anticipated that antibodies can recognize short peptides up to about 4-6 amino acid residues, which primary sequence maximally fits to the binding site of the antibody, good adjuvants obviously require considerably longer sequences as shown in the present invention. Therefore, successful development of a peptide-based adjuvant is the result of a fortunate combination of correct selection of a monoclonal antibody clone among a large number of possible clones with correct fit of the selected antibody in respect of exact molecular site in the original molecule (model of the artificial mimetics) that will trigger the immune response. The third embodiment of the present invention is that the mimetic peptides created against biological model molecules were not automatically reproduced, as has been anticipated in the prior art. Instead, we found that the undesired property (pyrogeneity) of the model molecule was not reproduced in the mimetic peptides. Therefore, there are several advantageous aspects in the present invention over the prior art.

We investigated a number of antibodies and testing methods for the screening of phage-display libraries. Monoclonal antibody (mAb) named E6/1.2 produced against GMDP was selected for the model studies. A multi-step screening procedure yielded only a single biologically active peptide structure from a random library of 15-mer peptides. The peptide was non-immunogenic and distinctly less pyrogenic than the model GMDP, which was unexpected. It will be appreciated by those skilled in the art that if the peptide length was allowed to be longer, more possible structures of given length would be obtained. However, it is beneficial to keep the length of the peptide relatively small for preventing the generation of antibodies against it.

There are examples of a compound having no direct structural similarity to a ligand, binding to the ligand-specific receptor or antibody and mimicking the biological activity of the ligand. For example, peptide mimics of carbohydrates interact with lectins and antibodies as described by Cunto-Amesty et al. (2001). These peptides competed with carbohydrates for the antibody/lectin binding site although the amino acid residues of the antibody/lectin interacting with the peptide may not be those involved in antibody/lectin-carbohydrate interaction. The phenomenon of mimicry gives promise for generating T-cell-dependent immune responses against T-cell-independent carbohydrate antigens. Carbohydrate-mimicking peptides and peptide-mimotope encoding DNA plasmids can be used for vaccination against pathogens and tumors as shown by Pincus in U.S. Pat. No. 6,444,787.

The present invention shows that it is possible to design procedures to create artificial peptide adjuvants which do not necessarily contain unusual amino acids and carbohydrates but which mimic microbial cell wall structures closely enough to have biological activities similar to the cell wall components. While several bioactive phage clones carrying optimally around 15-mer peptides were found, they all appeared to have the same sequences. Surprisingly, the identified peptide sequence involved no amino acid sequences specific to the original GMDP. Whereas the present invention was exemplified by mimicry to microbial cell wall components, based on the present disclosure, it will be apparent to those skilled in the art that similar artificial peptide adjuvants can be prepared to any known existing adjuvant.

A high-affinity IgG1 mAb E6/1.2 was produced against GMDP. The antibody is commercially available from Miomarket Ltd. Turku, Finland). It could bind equally well to GMDP-Lys and a GMDP analog with L-iGln residue substituted for D-iGln (LL-GMDP); for details, see Mareeva T. Yu. et al. (1993). Binding of GMDP to E6/1.2 mAb could be inhibited by high concentration of disaccharide GlcNAc-β(1-4)-MurNAc, but not by dipeptide Ala-D-iGln. Hence, the mAb binding was determined mainly by a carbohydrate moiety of the glycopeptide, and the configuration of the iGln residue did not affect the binding. It is to be noted that while mAb E6/1.2 was used in confirming the concept of the invention, there is no evidence that this antibody clone could select the best possible adjuvant peptide mimetics or that it imitates best the original model molecule. Because it is possible to find a multitude of antibodies for screening of phage-clones of peptides, it is, on the contrary, possible that other active peptides could be found which act as effective adjuvants. Furthermore, a slightly different antibody could be used to identify a peptide reproducing the pyrogeneity characteristics of the model molecule (GMDP).

Two phage display libraries of random peptides, namely 6-mer and 15-mer peptides, displayed at the N-terminus of pIII coat protein of fd bacteriophage, were studied in detail. Each library contained at least 2×10⁸ clones. Phage clones expressing peptides reacting with E6/1.2 mAb were isolated by four rounds of affinity selection. The interaction of phages with biotinylated mAb was carried out in solution followed by absorption of mAb-phage complexes on immobilized streptavidin. This approach facilitated access of antibodies to mimotope-bearing clones. We used acidic buffer to dissociate mAb-peptide complexes during the first round of selection, while GMDP was used during subsequent rounds in order to achieve specific elution of peptides competing with GMDP for E6/1.2 mAb binding. Each subsequent round was carried out with lower concentrations of both phage particles (ranging from 10¹³ cfu/ml at the first round to 10⁸ cfu/ml at fourth round) and mAb (from 1 μM to 1 nM). This selection scheme was advantageous for obtaining phage clones expressing peptides with the highest affinity. On the other hand, the elution with the ligand (GMDP) enabled selection of peptides most likely to bind to the combining site of the E6/1.2 mAb.

To evaluate the efficacy of enrichment with phage clones expressing E6/1.2 mAb-specific peptides at different rounds of selection, we plated on Petri dishes 1/100 bacterial cells infected with eluted phages. The number of colonies was counted. It was found that selection was efficient only in the case of 15-mer peptides. With the 6-mer peptide library, the number of bacterial colonies progressively dropped from the first to fourth selection round, and finally we were not able to find a single clone binding specifically to E6/1.2 mAb. Evidently hexapeptides could not adequately mimic the spatial structure of GMDP, or E6/1.2 mAb-specific clones were eliminated upon construction of the library.

When the 15-mer peptide library was screened, twenty clones were selected after the fourth round. The DNA was isolated and sequenced. Surprisingly, all clones had at the 5′ end of pIII fusion gene the identical sequence GGG GCT AGG GTT CCT CCG CGT TAT CAT GCT AAG ATT TCT CCT ATG GTG AAG GGG GCC GCT (SEQ ID NO: 1) which coded for oligopeptide RVPPRYHAKISPMVN (SEQ ID NO:2), the “RN-peptide”. A search of the NCBI Blast Protein database (all non-redundant GenBank CDS translations+PDB+SwissProt+PIR+PRF=1,326,269 sequences; 424,737,763 total letters; Request ID 1044381781-01359-978) did not reveal identical sequence in the NCBI Blast Protein database at http://www.ncbi.nlm.nih.gov/blast/Blast.cgi, although a homologous sequence (33% homology) was found in human U2 splicing factor (Kielkopf et al. 2001).

The sequence as found did not include peptide sequence fragments of GMDP, e.g. Ala-Gln or Ala-Glu. Neither could we find any homology with GlcNAc-mimicking peptide SFGSGFGGGY (SEQ ID NO: 3) described by Shikhman et al., (1994). E6/1.2 mAb apparently interacts with both carbohydrate and peptide moieties of GMDP, although interaction with the GlcNAc-MurNAc fragment seems to be of major importance. Without wishing to be bound by theory or mechanism, evidently RN-peptide shared the surface topology with the whole GMDP molecule rather than any part of its sequence. This necessitates that RN-peptide had to have a definite 3-D structure mimicking the original GMDP model in solution. However, RN-peptide shared no homology with other carbohydrate-mimicking peptides described up to now. In particular, it did not include aromatic amino acid repeats such as W/YXY (Valadon et al. 1996), WLY (Valadon et al. 1996), or YRY (Westerink et al. 1995). On the other hand, the characteristic feature of RN-peptide was the presence of the block consisting of two Pro, two Arg and two hydrophobic residues (Tyr, Val) within the N-terminal part of the peptide. Different carbohydrate mimotopes may reveal the same set of building blocks: aromatic, hydrophobic and hydrogen-bonding residues, as well as residues with the ability to position them appropriately in space. Most likely the Pro residues in the RN-peptide form some kind of β-turn or another type of short irregular structure, which orients the side chains of Arg and Tyr spatially, enabling interaction with E6/1.2 mAb.

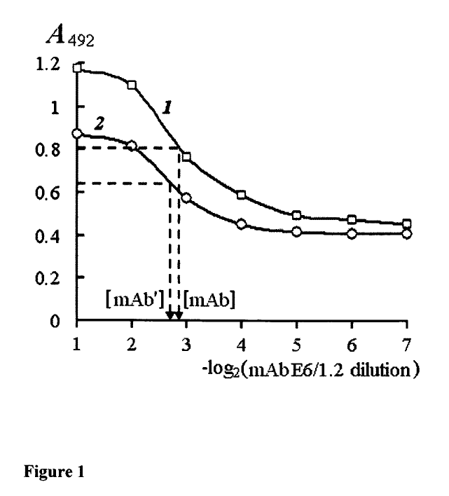

The affinity of binding of mAb E6/1.2 to selected phage clone expressing RN-peptide-pIII fusion protein was determined by indirect ELISA (FIG. 1). The affinity constant was 1.6×10⁷M⁻¹.

Different biological activities are characteristic of GMDP, including adjuvant activity, induction of non-specific resistance to infections, and the ability to induce Ia-antigen expression on murine macrophages (Khaidukov S. V. 1995). In order to evaluate the biological properties, we produced RN-peptide by solid-phase synthesis by Fmoc strategy. Two types of polymers were used, namely standard Wang's resin and TentaGel (Rapp Polymere, Germany). The synthesis carried out on Wang's resin yielded, after removal of protective groups, the free peptide which was used in biological studies after standard chromatographic purification. When hydrophilic TentaGel was used as solid support, the peptide remained bound to polymer after removal of protective groups enabling evaluation of its binding to E6/1.2 mAb by solid-phase ELISA. With this method, we demonstrated that synthesized peptide retained the ability of the phage particles bearing RN-peptide-pIII fusion protein to bind GMDP-specific mAb.

To study the adjuvant activity of RN-peptide, Balb/c mice were immunized with ovalbumin in saline in the presence of the RN-peptide, its analogs or GMDP, or without any adjuvant (control). The adjuvant was introduced on first immunization, whereas second immunization was carried out with half dose of OVA without adjuvant. It was found that RN-peptide strongly enhanced antibody response. Under optimal conditions the anti-ovalbumin antibody titer exceeded 6.5-7.5 times the titer of control serum. The optimal dose of RN-peptide depended on the dose of the antigen (Tables 1 and 2). When first immunization was carried out with 5-10 μg of OVA, antibody titer peaked at 1 μg/mouse of RN-peptide, whereas at 2.5 μg OVA the highest response was observed at 10 μg adjuvant dose. Hence, stronger antigenic stimulus required less adjuvant to obtain optimal results. At higher RN-peptide dose (100 μg/mouse) the anti-ovalbumin antibody titer diminished.

RN-peptide also enhanced antibody response to carbohydrate antigen. When low dose (0.2 μg) of capsular polysaccharide of Neisseria meningitidis serogroup C was administered i.v. in mice the anti-polysaccharide antibody titer increased considerably (Table 3). The optimal dose of peptide was higher than for the protein antigen ovalbumin. Taking into account that capsular polysaccharide C of Neisseria meningitidis is a world-recognized vaccine against meningococcal meningitis C, RN-peptide might be recommended in vaccination for reducing the dose of immunogen.

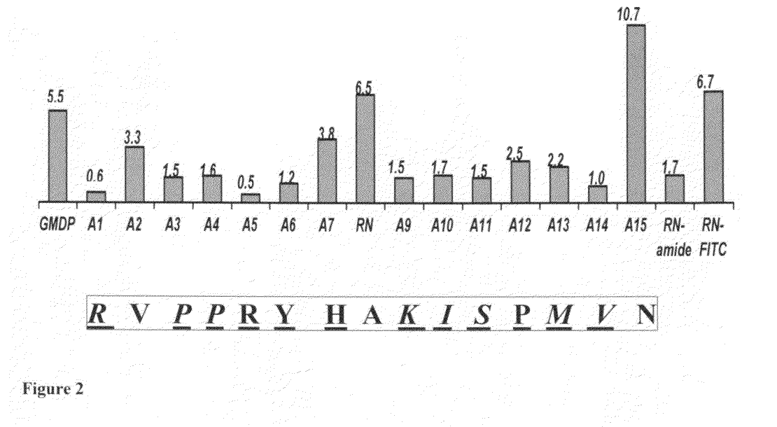

When peptides truncated from the N- or C-terminus were studied, the N-terminal portion of RN-peptide was shown to be crucial for the adjuvant activity. N-terminal decapeptide was adjuvant active, whereas deletion of the N-terminal Arg completely abrogated the adjuvant activity. On the other hand, C-terminal Asn could be deleted without substantial loss of activity. The smallest peptide retaining adjuvant effect of RN-peptide was heptapeptide (RH-peptide), although 100 times higher dose was required for maximal effect (Table 1). This data indicates that Arg, Pro and Tyr residues at N-terminus are of major importance for biological activity of RN-peptide. This was shown by step-by-step changing of amino acids in RN-peptide by Ala residues (FIG. 2). Substitution of Arg, Pro and Tyr residues at the N-terminus abolished or substantially reduced adjuvant activity. Most likely, positive charge at the N-terminal residue is crucial. In this case Arg can be replaced with Lys. It should be noted that certain other amino acids (Lys-9, Ile-10, Ser-11, Met-13 and Val-14) were also of major importance in the case of full-length peptide. Evidently, these residues assisted the N-terminal fragment to adopt the necessary conformation for optimal adjuvant activity. Taking into account that Ala has a non-polar side chain differing only in size from Ile and Val, we can assume that side chains of Ile-10 and Val-14 residues interact with other hydrophobic residues of RN-peptide. When Ala was introduced instead of C-terminal Asn, the activity of RN-peptide even increased. Evidently, the C-terminal residue can be modified in order to avoid hydrolysis of RN-peptide by carboxypeptidases. When a bulky aromatic group (FITC) was attached to the carboxyl terminus of RN-peptide through aminohexanoic acid spacer and Lys residue, a highly active compound was obtained, demonstrating the adjuvant index of 17.7 at 10 μg/mouse dose. Thus, as for muramyl- and glucosaminylmuramyl peptides, the introduction of a hydrophobic group has pronounced effect on the adjuvant activity.

Because of the presence of Arg and Lys residues, the RN-peptide is hydrolyzed by trypsin-like proteases. Many trypsin-like proteases are present in animal sera, reducing the serum half-life of the RN-peptide. Evidently, the degradation of peptide increases the dose of peptide required for in vivo adjuvant effect. To avoid this effect, the Arg residue at position 5 and the Lys residue at position 9 could be replaced with D-analogs or their side-chain amino group could be modified to increase the adjuvant activity.

The search for antibodies to the RN-peptide in the sera of mice immunized once or twice in phosphate-buffered saline (PBS) with peptide or ovalbumin in the presence of the peptide did not reveal any anti-RN-peptide antibodies. Hence, the humoral anti-RN-peptide response was not induced.

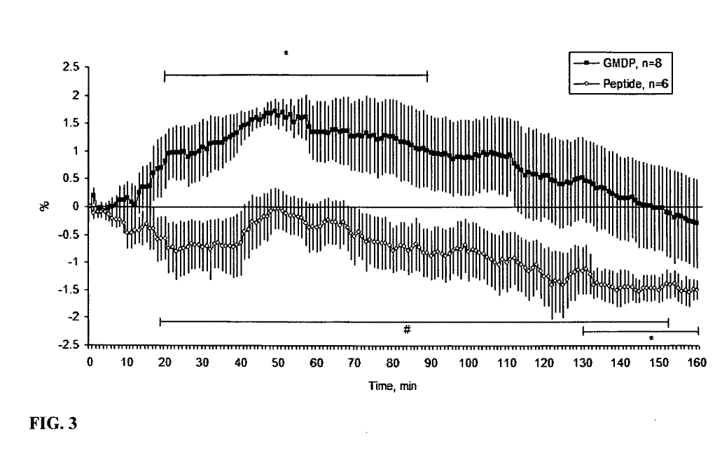

It has previously been shown that in vitro incubation with GMDP augmented MHC class II antigen expression on murine peritoneal macrophages (Khaidukov et al., 1995). Similar data was obtained for MDP-stimulated human monocytes by Heinzelmann et al. (2000). MDP was found to increase surface expression of HLA-DR, CD18, CD54 and CD86. Thus, upregulation of MHC class II antigen on monocytes/macrophages seems to be a characteristic feature of biologically active muramyl peptides. We studied the Ia-antigen-inducing activity of RN-peptide by stimulation in vitro of murine peritoneal macrophages. Flow cytometry was used to assess Ia-antigen expression. The number of Ia-positive cells in cultures treated with GMDP or RN-peptide increased as compared to the control. For both compounds, a dose-dependent, bell-type response was observed. In the case of RN-peptide, the response peaked at 1 μg/ml concentration, whereas the peak with GMDP was at 10 μg/ml. These doses of RN-peptide and GMDP induced an equal increase in Ia-positive cell numbers. The pyrogenicity of the peptide was studied in Wistar rats. Groups of 6 rats were injected i.p. with peptide in phosphate-buffered saline (PBS) or with an equi-molar dose of GMDP known to be pyrogenic. The dynamics of alteration of rectal temperature were monitored during 2.5 h. The increase in body temperature in the range 2.0-4.0% was observed in all animals receiving GMDP, whereas the injection of RN-peptide did not produce this effect. Hence, RN-peptide was non-pyrogenic, or at least distinctly less pyrogenic than GMDP (FIG. 3).

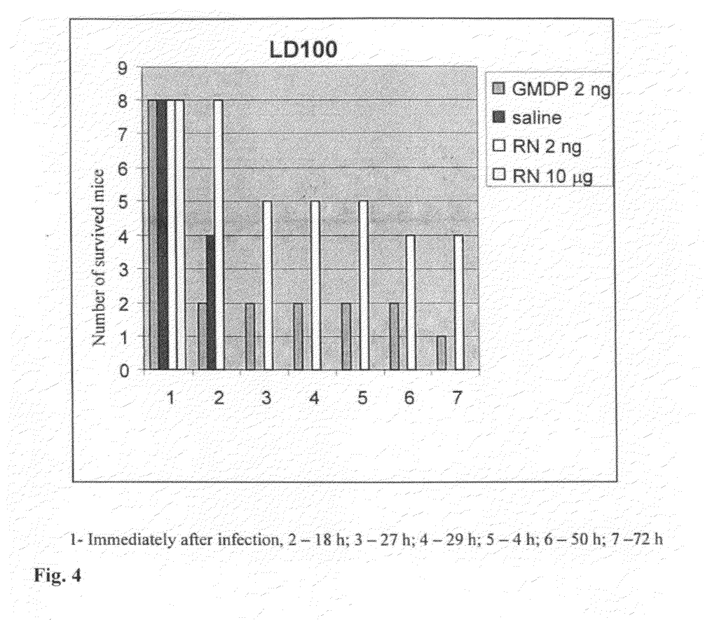

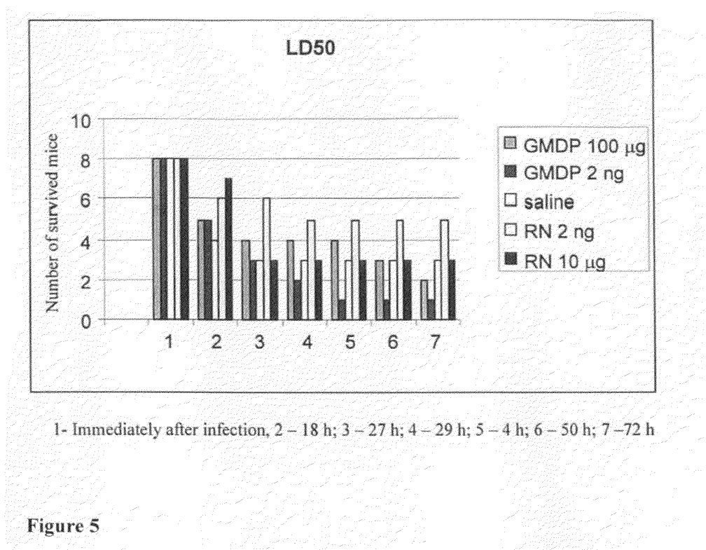

Muramyl peptides are known to stimulate in animals and humans a non-specific resistance to bacterial and viral infections. The ability of MDP and some of its derivatives to induce non-specific resistance to Klebsiella pneumoniae, Salmonella, Streptococcus pneumoniae, Pseudomonas aeruginosa, Candida albicans, Listeria monocytogenes, etc. have been documented (Lederer, 1988). GMDP was shown to protect mice against lethal challenge infection by E. coli and P. aeruginosa and to drastically decrease the virus content in mouse lungs upon infection with influenza virus Andronova and Ivanov (1991). These results have led to the recommendation that GMDP be used as an anti-infectious medicine for humans. Therefore we tested if RN-peptide could share with GMDP, not only adjuvant, but also anti-infectious activity. We used a murine model of infection with E. coli cells. The results demonstrate that substantial protection of mice is achieved from lethal challenge infection when mice are pretreated with RN-peptide before the infection (FIGS. 4-5).

Accordingly, it is apparent from this disclosure that we have identified peptides which mimic the antigenic structure of GMDP, and more importantly, which mimic GMDP's biological activity in inducing antigen-specific humoral and cellular immune responses, including but not limited to antibody formation and anti-bacterial activity. The compound according to this invention, however, did not mimic GMDP's pyrogenicity. RN-peptide or its analogs (truncated or with conservative substitutions) can be used for inclusion in vaccines as an adjuvant. In addition, certain amino acid modifications of the RN- and other peptides can be introduced. We have also observed that blocking of the N- and C-terminal ends of the peptides by suitable protecting groups can increase the biological activities, obviously by preventing quick disruption of the peptides. The adjuvant action of RN-peptide was further verified by the observations that the peptide itself functioned as an anti-infective substance.

One skilled in the art would realize that it would be possible to identify peptide mimetics for any adjuvant-active low molecular weight substance without deviating from the spirit of this invention. Different lengths of peptides in the phage display library can be used to optimize the activity and size of the de novo peptide adjuvant. One skilled in the art would also realize that with the same methods and principles as employed in the present invention, artificial peptide mimetics can be created to any adjuvant. The invention is described further below with non-limiting examples.

### EXAMPLE 1

### Monoclonal Antibodies Against GMDP and Selection of Peptides Binding to Antibodies

Materials. Monoclonal antibody E6/1.2 against GMDP was produced according to standard methods. The MKD6 hybridoma was a gift of Dr. P. Marrack (USA). Combinatorial libraries of hexa- and pentadecapeptides displayed on filamentous phage were a kind gift of Smith G. P et al. (the library described in Methods in Enzymol. (1993) 217, 228-257). The E. coli strain K91Kan was used for transfections. Balb/c and CBA mice and SPF Wistar rats were from the breeding house of the Pushchino Branch of the Shemyakin-Ovchinnikov Institute of Bioorganic Chemistry, Russia. Monoclonal antibody E6/1.2 is also available from BioMarket Ltd., Turku, Finland. The antibody was biotinylated with biotin N-hydroxysuccinimide ester (Bio-Rad, USA) according to the manufacturer's instructions.

Affinity selection of bacteriophages binding to E6/1.2 mAb. Streptavidin solution (10 μg/ml, 100 μl) in 0.1M Na-carbonate/bicarbonate buffer, pH 9.5, was introduced onto wells of 96-well micro-titer plate. The incubation was carried out overnight at 4° C. The plate was washed 5 times with PBS and 5 times with PBS containing 0.1% Tween-20. The mixture of 1% BSA and 5% horse serum in PBS (100 μl) was added, and the incubation was continued for another 2 h at 4° C. The plate was washed again. Phage display library (1013 TU, 100 μl) was mixed with biotinylated E6/1.2 mAb (bio-E6/1.2, 100 μl) at 15 μg/ml concentration for the first and second rounds, 1.5 μg/ml and 150 ng/ml for the third and forth rounds, respectively. After 1 h incubation at room temperature the mixture was introduced onto wells of microtiter plate coated with streptavidin. The plate was washed 5 times with PBS and 5 times with PBS containing 0.1% Tween-20. During the first round of selection, bound phages were eluted by 5-min incubation at room temperature with 100 μl/well of 0.2 M glycine-HCl, pH 2.0. Pooled eluate was immediately neutralized with 1/12 volume of 1M Tris. During the next rounds, phage particles were eluted with 1 μM GMDP by incubation at room temperature for 30 min.

Amplification of affinity-selected phages. Mid-log E. coli K91Kan cells (100 μl) were added to eluted phages. The cell suspension was incubated for 30 min at 37° C. without stirring and then transferred into 5 ml 2xYT medium containing 50 μg/ml kanamycin and 0.2 μg/ml tetracycline. The cells were grown for 1 h at 37° C. with vigorous aeration. Tetracycline was added to make final concentration of 20 μg/ml. To monitor the efficiency of the affinity selection, an aliquot of infected cells was plated on Petri dishes with 2YT medium, containing 50 μg/ml kanamycin and 20 μg/ml tetracycline and incubated overnight at 37° C. The number of colonies was then counted. Remaining cells were grown overnight at 37° C. with vigorous aeration. Individual colonies were picked up from Petri dishes after fourth round of affinity selection.

Purification of bacteriophages. Phage-containing E. coli culture, grown overnight, was centrifuged for 10 min at 8000 g. One fifth volume of PEG/NaCl solution (20% PEG 6000 and 2.5M NaCl) was added, and the incubation was carried out for 1 h at 4° C. The precipitate was pelleted by centrifugation and dissolved in 1/10 volume of TE buffer (10 mM Tris-HCl, pH 7.5, 1 mM EDTA, pH 8.0). The precipitation with PEG/NaCl was repeated one more time. The precipitate was dissolved in 1/100 volume of TE buffer, and insoluble material was spun down. The approximate concentration of phage particles was determined from the equation n=(A₂₆₉−A₃₂₀)·0.67·10¹³, where n is the number of phage particles per ml.

Isolation of DNA. Individual phage clones were used for isolation of single-stranded DNA. Magnesium chloride (10 mM final concentration) and pancreatic DNAse (5 μg/ml final concentration) were added to phage particles to remove bacterial DNA. After 30-min incubation at 37° C., EDTA was added to final concentration 5 mM, and successive extractions with equal volume of phenol, phenol-chloroform mixture and chloroform were carried out. Sodium acetate (0.3 M final concentration) and 2 volumes of ethanol were added. After 2 h incubation at −20° C., DNA was collected by centrifugation at 10,000 g for 15 min at 4° C. The precipitate was washed with 70% ethanol and dissolved in 10 μl TE buffer.

DNA sequencing was carried out according to Sanger using T7 Sequenase_{v 2.0} kit (Amersham) with fUSE ³²P primer complementary to gIII 1663-1680 sequence of wild type fd bacteriophage.

### EXAMPLE 2

### Characterization of the Selected Peptides

Peptide synthesis. All peptides were synthesized manually by the Fmoc/t-Bu technique. Wang Resin (Bachem, Switzerland) was loaded with Fmoc-Asn(Trt)-OH via diisopropylcarbodiimide-4-dimethylaminopyridine (DIC-DMAP) procedure (Heinzelmann et al. 2000). All couplings were performed with 2-(1H-benzotriazol-1-yl)-1,1,3,3-tetramethyluronium tetrafluoroborate (TBTU) in the presence of 1-hydroxybenzotriazol (HOBt) and N-methylmorpholine (NMM), at a ratio Fmoc-AA-OH:TBTU:NMM:HOBt=4:3, 6:4:2 equivalents with respect to polymer-bound amino acid. In parallel, the same sequence was assembled on TentaGel R (Rapp Polymere, Germany) bearing free amino groups to yield after final deprotection free 15-mer attached to biocompatible support directly suitable for AB binding studies. Coupling efficiency during polypeptide chain assembly was routinely monitored by Kaiser ninhydrine test and in case of incomplete reactions extra couplings were carried out. After completion of chain assembly and Fmoc-group removal peptide-resins were dried to constant weight. A mixture of trifluoroacetic acid with water, ethanedithiol and triisopropylsilan (94:2, 5:2, 5:1) was used for cleavage and/or complete deprotection at the final step. After standard post-cleavage work-up and lyophilization the crude peptide was purified by preparative RP HPLC.

Determination of affinity constants. Rabbit antibodies against M13 phage (100 μl) were introduced into wells of 96-well microtiter plate in 0.1 M sodium bicarbonate buffer, pH 8.6, at 5 μg/ml concentration. Incubation was carried out overnight at 4° C. followed by incubation for 2 h at room temperature with 1% gelatin in PBS in order to block remaining binding sites. The plate was washed 5 times with PBS and 5 times with PBS/0.1% Tween-20 (PBS-T). The same washing procedure was applied after each incubation step. Phage particles (10¹¹ PU/ml-C₀ or 5×10¹⁰ PU/ml-C₀/2) in PBS were added. After 2 h incubation at room temperature unbound phage particles were removed. Serial two-fold dilutions of E6/1.2 mAb in PBS-T (100 μl) were added starting from 5 μg/ml. Incubation was carried out for 2 h at room temperature. The plate was washed and incubated with HRP-conjugated rabbit anti-mouse antibodies. The bound conjugate was visualized with OPD. The affinity of binding of mAb E6/1.2 to selected phage clone expressing RN-peptide-pIII fusion protein was determined by indirect ELISA (FIG. 1). The affinity constant was 1.6×10⁷M⁻¹.

### EXAMPLE 3

### RN Peptide and its Truncated Analogs have Adjuvant Activity

Adjuvant activity. Groups of 5 Balb/c mice were administered i.p. with 2.5-10 μg ovalbumin (Sigma, USA) per mouse in 200 μl PBS. RN-peptide (0.1-100 μg) or GMDP (1-100 μg) was introduced simultaneously with ovalbumin on first immunization. The second immunization was carried out 28 days later with half dose of ovalbumin (1.25-5.0 μg/200 μl) without adjuvant. Control mice received ovalbumin twice without adjuvant. Three days after the second immunization mice were bled from the tail vein. Immunization with N. meningitidis serogroup A capsular poly-saccharide (demonstrating the use of peptide adjuvants in a vaccine) was carried out i.v. with 0.05 μg of antigen which was administered once. Control mice received polysaccharide without any adjuvant. Five and ten days after immunization mice were bled.

The antibody titer was evaluated by solid-phase ELISA as described by Douillard and Hoffman (1983) Enzyme-linked immunosorbent assay for screening monoclonal antibody production using enzyme-labeled second antibody. The antigen (ovalbumin or polysaccharide), 5 μg/ml in bicarbonate buffer, pH 8.6 (100 μl), was introduced into wells of 96-well microtiter plate. Incubation was carried out overnight at 4° C. The plate was washed 5 times with PBS and 5 times with PBS/0.1% Tween-20 (PBS-T). Remaining binding sites were blocked with 1% gelatin in PBS (2 h, room temperature). After washing 100 μl of mouse serum (1:100 dilution) was added, and serial three-fold dilutions were made. Bound antibodies were visualized by incubation with Horse-Radish-Peroxidase (HRP)-conjugated rabbit anti-mouse antibodies and OPD as the chromogenic substrate. The antibody titer was the last dilution, which showed A₄₉₂ value exceeding two times the A₄₉₂ of control serum taken from mice, immunized without adjuvant. The results are shown in Tables 1 and 2.

TABLE 1
Adjuvant effect of RN-peptide on antibody
response to ovalbumin in mice

| | Anti-ovalbumin | Adjuvant |
|---|---|---|
| Adjuvant and its dose | antibody titer* | index |
| No adjuvant | 2450 ± 100 | 1 ± 0.04 |

RN-peptide

| | | | |
|---|---|---|---|
| 0.1 | μg | 2250 ± 100 | 0.92 ± 0.04 |
| 1 | μg | 4300 ± 150 | 1.75 ± 0.06 |
| 10 | μg | 19000 ± 200 | 7.55 ± 0.06 |
| 100 | μg | 2000 ± 20 | 0.8 ± 0.008 |

| | | |
|---|---|---|
| GMDP, 10 μg | 5800 ± 150 | 2.37 ± 0.06 |

*Mice were immunized twice with 2.5 μg (first immunization) and 1.25 μg ovalbumin. RN-peptide or GMDP was administered only upon first immunization.

TABLE 2
Adjuvant effect of truncated RN-peptide analogs
on antibody response to ovalbumin in mice.

| | | Anti-ovalbumin | |
|---|---|---|---|
| Adjuvant and dose | | antibody titer | Adjuvant index |
| Control (no adjuvant) | | 6000 ± 200 | 1.0 ± 0.03 |
| RN-peptide | | | |
| (SEQ ID NO: 2) | | | |
| 1 | μg | 43500 ± 400 | 7.3 ± 0.08 |
| 10 | μg | 15700 ± 280 | 2.6 ± 0.05 |
| 100 | μg | 5400 ± 200 | 0.9 ± 0.03 |
| RV-peptide (14-mer, | | | |
| truncated from C-terminus) | | | |
| (SEQ ID NO: 8) | | | |
| 1 | μg | 37800 ± 350 | 6.3 ± 0.06 |
| 10 | μg | 8400 ± 240 | 1.4 ± 0.04 |
| 100 | μg | 4800 ± 180 | 0.8 ± 0.03 |
| R-I-peptide (10-mer, | | | |
| truncated from C-terminus) | | | |
| (SEQ ID NO: 9) | | | |
| 1 | μg | 24000 | 6.3 ± 0.06 |
| 10 | μg | 19200 | 3.2 ± 0.09 |
| 100 | μg | 25200 | 4.2 ± 0.05 |
| R-H-peptide (7-mer, | | | |
| truncated from C-terminus) | | | |
| SEQ ID NO: 10) | | | |
| 1 | μg | 4000 | 0.7 ± 0.03 |
| 10 | μg | 13900 | 2.3 ± 0.05 |
| 100 | μg | 51900 | 8.6 ± 0.09 |
| VN-peptide (14-mer, | | | |
| truncated from N-terminus) | | | |
| SEQ ID NO: 11) | | | |
| 1 | μg | 5400 ± 200 | 0.9 ± 0.03 |
| 10 | μg | 8400 ± 230 | 1.4 ± 0.04 |
| 100 | μg | 4200 ± 150 | 0.7 ± 0.03 |
| GMDP | | | |
| 1 | μg | 4000 ± 150 | 0.7 ± 0.03 |
| 10 | μg | 7900 ± 200 | 1.3 ± 0.03 |
| 100 | μg | 64800 ± 500 | 10.8 ± 0.08 |

*Mice were immunized twice with 10 μg (first immunization) and 5 μg ovalbumin. RN-peptide or GMDP was administered only upon first immunization.

Immunization with capsular polysaccharide of N. meningitidis serogroup C was carried out in CBA mice. Groups of 5 mice were administered i.v. with polysaccharide C (0.2 μg) in saline in the presence of 1-100 μg of RN-peptide. Control mice were immunized with optimal dose (2 μg/mouse) of polysaccharide. Four days later mice were bled from tail vein and the antibody titer was evaluated by passive agglutination test using commercial erythrocyte diagnostic kit.

TABLE 3
Antibody response to N. meningitidis
serogroup C capsular polysaccharide

| Group | | Anti-polysaccharide C |
|---|---|---|
| of mice | Immunogen | antibody titer |
| 1 | C polysaccharides, 0.2 μg | <1:2 |
| 2 | C polysaccharides, 0.2 μg + | 1:4 |
| | RN-peptide, 1 μg | |
| 3 | C polysaccharides, 0.2 μg + | 1:8 |
| | RN-peptide, 10 μg | |
| 4 | C polysaccharides, 0.2 μg + | 1:32 |
| | RN-peptide, 100 μg | |
| 5 | C polysaccharides, 2 μg (optimal dose) | 1:80 |

Hence, both in the case of protein and polysaccharide antigen, RN-peptide demonstrated its adjuvant effect.

### EXAMPLE 4

### RN Peptide is not Immunogenic

Immunogenicity of the peptide. Groups of Balb/c mice (5 mice) were immunized with 1-100 μg RN-peptide in PBS i.p. twice with 10-day interval. The anti-peptide antibody titer was estimated in pooled sera by solid-phase ELISA using MaxiSorb plates (Nunc, Denmark) coated with RN-peptide (10 μg/ml). The anti-peptide antibody titer was compared with the titer of normal mouse serum. E6/1.2 mAb was used as a positive control.

For all groups of mice the anti-RN-peptide antibody titer did not exceed the titer of native mice. Hence, two immunizations with RN-peptide did not induce humoral immune response to peptide.

### EXAMPLE 5

### RN Peptide has the Ability to Induce MHC Class II Gene Expression in Macrophages

Ia-antigen-inducing effect. Balb/c mice peritoneal mononuclear cells (PMC) were isolated by Histopaque density gradient centrifugation. PMC or WEHI-3 cells were incubated with RN-peptide (0.1-10 μg/ml) or GMDP (positive control) or saline (negative control) in RPMI 1640 medium, supplemented with 5% fetal calf serum upon rotary mixing for 18 h at 37° C. Cells were stained on ice bath with MKD6 anti-Ia^{d} mAb (supernatant from the in vitro culture, 1:10 dilution) and FITC-labeled anti-mouse immunoglobulin (Sigma, USA, 1:300 dilution), and then with phycoerythrin-labeled F4/80 mAb (Caltag, USA, 0.2 μg/100 μl). Ia antigen expression was assessed by flow cytometry using EPICS “Elite” (Coulter, USA).

The overnight treatment of murine mononuclear cells with RN-peptide or GMDP resulted in increase in Ia-positive cell number. For both compounds at optimal dose (0.1 μg/ml for RN-peptide and 10 μg for GMDP) 10% increase was observed. Similar results were obtained when myelomonocytic WEHI-3 cells were used as targets. Hence, similarly to parent GMDP, RN-peptide demonstrated the ability to induce MHC class II gene expression in macrophages.

### EXAMPLE 6

### RN Peptide has Lower Pyrogenicity than GMDP

The pyrogenic effect SPF female Wistar rats were injected i.p. with a peptide (1.01 mg/kg; 5.74×10⁻⁷ mole/kg) in PBS or with equal dose of GMDP (0.4 mg/kg; 5.74×10⁻⁷ mole/kg). Rectal temperature was recorded every 5 minutes for 160 minutes using PowerLabe/4sp system with an electronic thermometer (MLT1403 Rectal Probe for Rats, AD Instruments USA). All data were quantified (mean±SEM) and compared by the variance analysis. Results in FIG. 2 show that the artificial RN-peptide has distinctly lower pyrogenicity than the parent GMDP.

### EXAMPLE 7

### RN Peptide has Anti-Infective Mode of Action

Groups of Balb/c mice (20-22 g) were administered i.p. with different doses of RN-peptide (1 ng-10 μg) or GMDP (1 ng-10 μg) in saline. Control mice received saline. Mice were infected i.p. 7 days later with E. coli strain K12 for which LD100 and LD50 were 2×10⁸ and 1×10⁸ cells/mouse, respectively. Survival of mice was assessed during three days. FIGS. 4 and 5 show that RN-peptide has a substantial anti-infective mode of action, i.e., it generates faster response to an infection.

### EXAMPLE 8

### RN-Peptide Protects Mice from Lethal E. coli Infections

Groups of Balb/c mice (20-22 g) were administered i.p. with RN-peptide, or GMDP, or saline (control), and then infected i.p. with E. coli strain TG-1 cells. Two doses of E. coli cells were used—1×10⁸ (LD50) and 2×10⁸ (LD100). Mice were infected one day (experiment A) or seven days (experiment B) after injection of the immunomodulator. Survival of mice was assessed during three days. Results showing that RN-peptide protects mice from lethal doses of E. coli infections (Table 4).

TABLE 4
Effect of RN-peptide on survival of mice against
lethal doses of bacterial infection.

| | |
|---|---|
| Hours after infection | Number of |

| | | | | | |
|---|---|---|---|---|---|
| Immunomodulator | 0 | 24 | 28 | 72 | survivors |

Experiment A
1 × 10⁸ E. coli cells (LD50)

| | | | | | |
|---|---|---|---|---|---|
| RN Peptide, 2 ng | 6 | 6 | 5 | 5 | 5 |
| GMDP, 2 ng | 6 | 2 | 2 | 2 | 2 |
| Control | 6 | 5 | 3 | 2 | 2 |

2 × 10⁸ E. coli cells (LD100)

| | | | | | |
|---|---|---|---|---|---|
| Peptide, 2 ng | 6 | 5 | 5 | 2 | 2 |
| GMDP, 2 ng | 6 | 2 | 2 | 1 | 1 |
| Control | 6 | 2 | 0 | | 0 |

Experiment B
1 × 10⁸ E. coli cells (LD50)

| | | | | | |
|---|---|---|---|---|---|
| Peptide, 2 ng | 6 | 6 | 5 | 4 | 4 |
| GMDP, 2 ng | 6 | 5 | 2 | 2 | 2 |
| Control | 6 | 5 | 5 | 2 | 2 |

2 × 10⁸ E. coli cells (LD100)

| | | | | | |
|---|---|---|---|---|---|
| Peptide, 2 ng | 6 | 3 | 3 | 1 | 1 |
| GMDP, 2 ng | 6 | 3 | 2 | 0 | 0 |
| Control | 6 | 3 | 2 | 0 | 0 |

### EXAMPLE 9

### Methods for Production of the Adjuvant for Pharmaceutical Use

According to one preferred embodiment the peptides according to this disclosure are artificially synthesized for example by solid phase technique. Such synthetic peptides are safe for pharmaceutical use due to lack of possible harmful impurities originating from the microbial host cells.

Another preferred embodiment comprises of expressing the peptide molecule in a host cell as a multimeric polypeptide encoded by a genetic construct comprising multiple copies of a nucleotide sequence encoding subunits of the adjuvant peptide. The multiple copies are linked in the construct in a manner that they are expressed as a single multimeric polypeptide. A suitable host cell may be A prokaryotic or eukaryotic cell, and may involve bacterial, yeast, or mammalian expressions systems. Preferably the construct contains sequences that are susceptible to proteolysis and therefore multimeric polypeptide would be cleaved into active peptide sequences prior to or once administered to patient.

### REFERENCES

1. Ellouz, E., et al. (1974) Biochem. Biophys. Res. Commun. 59, 1317-1325.
2. Lederer, E. (1988) in: Advances in Immunomodulation, (Bizzini, B. and Bonmassar, E., eds), Pythagora Press, Roma-Milan, 9-36.
3. Rostovtseva, L et al. (1981) Bioorg. Khim. (Moscow). 7, 1843-1858.
4. Andronova T., et al. (1991) Sov. Medical Reviews. D. Immunology (Harwood Academic Publishers), 4, 1-63.
5. Ivanov V. T. et al. (1996) Immunologiya (Moscow), 4-6.
6. Sparks, A. B., et al. (1996) Phage display of peptides and proteins. A Laboratory Manual. (Kay, B. K. et al. eds.) Academic Press, 227-254.
7. Mareeva T. Yu. et al. (1993) Bioorg. Khim. (Moscow) 19, 555-561.
8. Cunto-Amesty G et al. (2001) Int. Rev. Immunol. 20, 157-180.
9. Kieber-Emmons T et al. (2000) J Immunol., 165, 623-7.
10. Smith G. P et al. (1993) Methods in Enzymol. 217, 228-257.
11. NCBI Blast Protein database at http://www.ncbi.nlm.nih.gov/blast/Blast.cgi.
12. Kielkopf C L et al. (2001) Cell, 106, 595-605.
13. Shikhman A R et al. (1994) J. Immunol. 153, 5593-5598.
14. Valadon P et al. (1996) J. Mol. Biol. 261, 11-22.
15. Hoess R., et al. (1993) Gene. 128, 43-49.
16. Beatty, J. D. et al. (1987) J. Immunol. Methods, 100, 173-179.
17. Westerink M A et al. (1995) Proc. Natl. Acad. Sci. 92, 4021-4025.
18. Rose G D et al. (1985) Adv. Protein Chem. 37, 1-109.
19. Khaidukov S. V. et al. (1995) Int. J. Immunopharmacol., 17, 903-911.
20. Mole, S. E. et al. (1987) DNA cloning. A practical approach to (Edited by Glover, D. M.), IRL Press, 202
21. Heinzelmann M. et al. (2000) Immunopharm. 48, 117-128.
22. Chan, W. C et al. (2000) In: Fmoc Solid Phase Peptide Synthesis. A Practical Approach. (Chan, W. C. et al, Eds.). Oxford University Press. New York, 41-76.
23. Douillard J Y et al. (1983) Methods in Enzymol. 92, 168-174
24. Metzler, D. E, Biochemistry, 2003, Second Edition, Academic Press. p. 1859

1. A method of generating novel peptide adjuvant molecules, said method comprising the steps of:
a. generating one or more antibodies against a template molecule with adjuvant properties;
b. contacting a peptide-displaying library with one or more of said antibodies;
c. isolating one or more peptide displaying entities having a displayed peptide which binds to one or more of said antibodies;
d. selecting from all said peptide displaying entities isolated in step (c) one or more peptides or peptide fragments to which one or more of said antibodies bind; and
e. expressing the selected peptides or peptide fragments in host cells or synthesizing the peptide or peptides chemically.

2. The method according to claim 1 wherein the template molecule is N-acetylglucosaminyl-β1-4-N-acetylmuramyl-alanyl-D-isoglutamine (GMDP).

3. The method of claim 1, wherein the peptide is expressed in a host cell as a multimeric polypeptide comprising multiple copies of the peptide.

4. A peptide molecule generated by the method of claim 1, said peptide molecule further retaining or exceeding the adjuvant activity of the template molecule.

5. The peptide molecule of claim 4, wherein the peptide molecule does not retain undesirable activities of the template molecule

6. The peptide molecule of claim 5, wherein the undesirable activities of the template molecule is pyrogenicity.

7. The peptide molecule of claim 33, wherein the peptide has an amino acid sequence selected from the group consisting of SEQ ID NO:2, SEQ ID NO:4, SEQ ID NO: 8, SEQ ID NO: 9, and SEQ ID NO:10.

8. A peptide molecule having adjuvant activity at least equal to that of N-acetylglucosaminyl-β1-4-N-acetylmuramyl-alanyl-D-isoglutamine (GMDP), and said peptide molecule specifically binding to an antibody generated against GMDP.

9. The peptide molecule of claim 8, wherein the peptide has an amino acid sequence according to SEQ ID NO:5, and aminoterminal end of the sequence is attached to a positively charged amino acid, a positively charged protecting group, or both, and carboxyterminal end of the sequence is optionally attached to one or several amino acids, one or more hydrophobic groups, a fluorescent molecule, peptide sequence according to SEQ ID NO: 6, peptide sequence according to SEQ ID NO: 7, or combinations and variations thereof.

10. The peptide molecule of claim 9, wherein the peptide has an aminoacid sequence selected from a group consisting of SEQ ID NO:2 SEQ ID NO:4 SEQ ID NO:8 SEQ ID NO:9 and SEQ ID NO:10.

11. The peptide molecule according to claim 8, wherein the amino acid sequence is a truncated or chemically modified from of SEQ ID NO: 2 and comprising at least seven amino acid residues. (this is now a repetition of claim 10, but we can leave for time being)

12. The peptide molecule according to claim 11, wherein the amino acid sequence of the peptide is selected from the group consisting of SEQ ID NO:8, SEQ ID NO:9 and SEQ ID NO:10. (also repetitive now but we can leave it now as is)

13. The peptide molecule according to claim 12, wherein Arg-residue in position 3 and/or Lys-residue in pos 5 are replaced with D-analogs.

14. A peptide molecule having adjuvant activity at least equal to that of N-acetylglucosaminyl-β1-4-N-acetylmuramyl-alanyl-D-isoglutamine (GMDP), said peptide being produced by a process comprising the steps of:
a) generating one or more antibodies against GMDP,
b) contacting a peptide-displaying library with one or more of said antibodies,
c) isolating one or morel peptide displaying entities having a displayed peptide which binds to one or several of said antibodies,
d) selecting from all said peptide displaying entities isolated in step (c) one or more peptides or peptide fragments to which one or several of said antibodies bind, and
e) expressing the selected peptides in host cells or chemically synthesizing the peptides.

15. The peptide according to claim 14, wherein the peptide has an amino acid sequence according to SEQ ID NO:5, and aminoterminal end of the sequence is attached to a positively charged amino acid, a positively charged protecting group, or both, and carboxyterminal end of the sequence is optionally attached to one or several amino acids, one or more hydrophobic groups, a fluorescent molecule, peptide sequence according to SEQ ID NO: 6, peptide sequence according to SEQ ID NO: 7, or combinations and variations thereof.

16. An isolated nucleic acid molecule encoding a peptide having an amino acid sequence selected from the group consisting of SEQ ID NO:2, SEQ ID NO: 4 and SEQ ID NO:5.

17. The isolated nucleic acid molecule according to claim 16, wherein the nucleic acid sequence is according SEQ ID NO: 1, and said nucleic acid molecule encodes a peptide having amino acid sequence of SEQ ID NO: 2.

18. A plasmid comprising the nucleic acid of claim 16.

19. A recombinant cell comprising the nucleic acid of claim 16.

20. A method for increasing immune response formation against an antigen, said method comprising administration of a peptide having an amino acid sequence according to SEQ ID NO:5, and aminoterminal end of the sequence is attached to a positively charged amino acid, a positively charged protecting group, or both, and carboxyterminal end of the sequence is optionally attached to one or several amino acids, one or more hydrophobic groups, a fluorescent molecule, peptide sequence according to SEQ ID NO: 6, peptide sequence according to SEQ ID NO: 7, or combinations and variations thereof.

21. The method of claim 20, wherein the amino acid sequence is selected from a group consisting of SEQ ID NO: 2, SEQ ID NO:4, SEQ ID NO:8, SEQ ID NO:9 and SEQ ID NO:10.

22. The method of claim 21, wherein the antigen is a carbohydrate antigen.

23. The method of claim 22, wherein the antigen is capsular polysaccharide C of Neisseria meningitis.

25. A composition comprising a peptide adjuvant identified according to the method of claim 1.

26. A composition comprising the peptide molecule according to claim 14.

27. A vaccine comprising a peptide adjuvant identified according to the method of claim 1.

28. A vaccine comprising the peptide molecule according to claim 14.

29. A method for preventing or reducing infection by a pathogen, said method comprising administering to a subject at risk of the infection an effective amount of the peptide identified according to the method of claim 1.

30. A method for preventing or reducing infection by a pathogen, said method comprising administering to a subject at risk of the infection an effective amount of the peptide of claim 14.

31. The method according to claim 29 further comprising co-administration of an antigen from said pathogen.

32. The method according to claim 30 further comprising co-administration of an antigen from said pathogen.

33. The peptide molecule of claim 6, wherein the peptide has an amino acid sequence according to SEQ ID NO:5, and aminoterminal end of the sequence is attached to a positively charged amino acid, a positively charged protecting group, or both, and carboxyterminal end of the sequence is optionally attached to one or several amino acids, one or more hydrophobic groups, a fluorescent molecule, peptide sequence according to SEQ ID NO: 6, peptide sequence according to SEQ ID NO: 7, or combinations and variations thereof.

34. The peptide molecule of claim 15, wherein the peptide has an amino acid sequence selected from the group consisting of SEQ ID NO: 2, SEQ ID NO:4, SEQ ID NO:8, SEQ ID NO:9 and SEQ ID NO:10.
